(19) United States
(12) Patent Application Publication
(10) Pub. No.: US 2004/0265360 A1
(43) Pub. Date: Dec. 30, 2004

(54) Ingestible composition

(76) Inventor: David Venturi, Oakville (CA)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/825,213

(22) Filed: Apr. 16, 2004

(30) Foreign Application Priority Data
Aug. 26, 2002 (SE) 0202529-4
Aug. 26, 2003 (WO) WO02004/017764

(51) Int. Cl. A61K 35/78; A61K 47/00

(52) U.S. Cl. 424/439; 424/757; 424/765

(57) ABSTRACT

The present invention provides an ingestible composition comprising a plurality of components consisting of fat, carbohydrates, proteins, vitamins and minerals, wherein relative amounts of the components of the composition are chosen such that an intake of said product reduces the effects of metabolic syndrome.

### PRIORITY CLAIM

The present application claims priority from Swedish Patent Application 0202529-4 filed Aug. 26, 2002, and PCT Patent Application Publication Number WO2004/017764, filed Aug. 26, 2003, the contents of each of which are incorporated herein by reference.

1. Field of the Invention

The present invention relates generally to ingestible compositions and more particularly relates to an ingestible composition for metabolic syndrome.

2. Background of the Invention

Even though the importance of balanced diets has been recognized by dietitians and scientists as well as by the general public for a long time, the maintenance of a healthy diet is not only a persisting but also a growing problem in modern society. With today's accelerated pace of living, it has become increasingly difficult for ordinary people to find the time to prepare meals ensuring a daily intake of nourishment essential to the human body.

The lack of time makes many people choose fast food alternatives, which often are fattening or have an inadequate nutritive value, or even skip entire important meals such as breakfast.

A further problem occurs in the composing of the food components. Even though there is a high awareness in the general public of which essential nutritive components should be included in an ordinary meal, very often in daily life it becomes difficult to estimate the amount of nutrients in the different ingredients constituting the meal. As the different food components are known to interact with each other, the total nutritive effect of the meal may be furthermore difficult to estimate.

Especially the effect of the absorbed food on the blood glucose level has shown to be of great importance for the well-being of humans. When food is eaten, digested and absorbed into the blood stream, a corresponding rise can be detected in the blood glucose level. Poorly balanced diets often include carbohydrates which, when converted to glucose, induce a rapid response of insulin. The so-called insulin spikes lead to a rapid storage of sugar, and subsequently also fat, into the cells, thus giving rise to an unnecessary amount of energy being stored in the body. Accordingly, shortly after the first meal, the blood glucose level in the blood is considerably reduced by the correspondingly rapid action of insulin, and the body signals for more glucose with feelings of hunger, tiredness, lack of concentration etc.

Because of the direct need for an increase of the blood glucose level, a craving for foodstuffs with a high sugar content is induced, again starting a new cycle of drastic fluctuations in the blood glucose level, when provided with further glucose. These swift fluctuations in the blood glucose level leave the consumer with temperamental ups and downs, and invite the consumer to a habit of constant eating which also becomes a problem in controlling the weight, and furthermore also will add fat to the heart and the blood system.

It is also important to note that in order to satisfy feelings of hunger, not all foodstuffs have proven to have a long term satiating effect. As the hungry consumer strives to balance the blood glucose level, a further problem occurs in the difficulty of choosing foodstuffs able to provide a satiety for a prolonged period of time.

The maintenance of an even level of insulin in the blood has further importance; elevated levels of insulin in the blood are associated with a lowered degree of efficacy in the tissue, i.e., insulin resistance. This condition is known to have an adverse effect on several important and common diseases, such as diabetes, hypertension, arteriosclerosis and hyperlipidemia, and obesity.

In order to compensate deficiencies in the daily diet, there are a vast number of food supplements available on the market. The purpose of these food supplements is to provide the consumer with a daily dosage of essential food nutrients, which are assumed to be lacking in the ordinary food consumption.

Commonly the consumer completes an inadequate daily meal with minerals and vitamins essential to the body. In addition, some people use different supplements in order to optimise their physical condition, such as products for muscle building.

However, this course of action to attain a complete meal in respect to the needs of the body is associated with certain drawbacks. Normally, it is complicated to estimate the amount of nutrients in a meal. As mentioned earlier, certain food components interact with each other so that the nutritive value of the overall meal may actually differ from the sum of the nutritive values of the individual components. When food supplements are being added, this estimation becomes even more difficult to calculate.

The consumer is furthermore provided with several food products which can replace entire meals. However, these products rarely contain all the essential components for use over a prolonged period. Most of these products are in fact intended to be used only until a certain purpose is achieved, for example, different slimming products, products for muscle building, and nutrient solutions for patients suffering from different diseases.

Because of the short time usage, these products are more often focused on solving an immediate problem and are thus not suitable for, or required to, being a replacement of all or part of a normal diet. Furthermore, a large number of the meal replacement products available on the market are composed in such a manner that a balanced physical effect on the human body system fails to appear, especially a maintenance of a stable constant blood glucose level.

Metabolic syndrome is used to describe a set of general set of risk factors identifiable, in at least some members of the population, as increasing the risk of heart attack, stroke, diabetes and certain other conditions. Metabolic syndrome is generally characterized by an elevated concentration of insulin in the blood, with the consequence of a more or less pronounced insulin resistance. Some of the known risk factors associated with the metabolic syndrome are high blood pressure, high levels of LDL cholesterol, high levels of triglycerides, obesity and type two diabetes. Metabolic syndrome is also associated with other medical disorders, including gastroesophageal reflux, snoring with bad night sleep, hyperplasia of the prostate in men and polycystic ovaries in women. For further information, see Davidson's Principle's and Practice of Medicine, 18th edition, 1999 (p. 475-477), the contents of which are incorporated herein by reference.

The cause of metabolic syndrome is not precisely known, although it is suggested by researchers that it is caused by a combination of genetic makeup and lifestyle, including the types of food and level of physical activity.

Prevention and/or treatment of metabolic syndrome have thus been suggested to include weight loss, physical exercise and a controlled diet (www.heartcenteronline.com). The most common drugs used in the treatment of the syndrome include cholesterol-lowering statins, antihypertensive drugs, drugs that reduce the gastric acid production and drugs to reduce the blood sugar level. However, the prevalence of metabolic syndrome suggests that these prevention and treatment programs can benefit from further improvements.

There is a need for an ingestible composition that provides the user with at least part of a complete balanced meal comprising food components for the body and which can be used for treatment or at least amelioration metabolic syndrome. It is also desirable to be able to offer an ingestible composition that may be used for a longer period of time without causing a lack or an excess of certain components in the human body.

### SUMMARY OF THE INVENTION

It is therefore an object of the present invention to provide an ingestible composition for use in association with metabolic syndrome that obviates or mitigates at least one of the disadvantages of the prior art.

In an aspect of the invention, there is provided a dry food composition product for mixing with a drinkable liquid, said product comprising fat, carbohydrates, proteins, vitamins and minerals, which food composition product is intended to replace an entire meal, characterized in that the relative amounts of said components of said food composition product are chosen such that an intake of said food composition product provides the consumer with a stable blood glucose level.

The product can be characterized in that the product weight ratio between carbohydrates, proteins and fat is of the magnitude of 0.8-2.0:1:0.1-0.4, respectively, wherein the carbohydrates are chosen from the group of foodstuffs having a low glycemic index.

The product can be characterized in that the product weight ratio between carbohydrates, proteins and fat is of the magnitude of 0.8-2.0:1:0.1-0.3, respectively, wherein the carbohydrates are chosen from the group of foodstuffs having a low glycemic index.

The product can be characterized in that the carbohydrates are derived from leguminous plants, preferably yellow peas, and rosaceous plants, preferably apples and rose hips.

The product can be characterized in that at least 15% of the overall content is derived from yellow peas, at least 10% from apples, and at least 5% from rose hips.

The product can be characterized in that the product is balanced so that the overall meal has GL-value below 20, preferably below 10.

The product can be characterized in that the carbohydrates are in the form of simple sugars from the group consisting of glucose, saccharose, fructose, maltose and lactose.

The product can be characterized in that the sugars are present in a content of 2.1-2.5%, preferably about 2.3%, glucose, 1.8-2.2%, preferably about 2.0%, saccharose, 4.0-4.8%, preferably about 4.4% fructose, <0.03-0.05%, preferably about <0.04%, maltose and 12.9-15.7%, preferably about 14.3% lactose.

The product can be characterized in that a ready to drink serving has either a low lactose content or alternatively is completely free from lactose.

The product can be characterized in that it comprises 7-12% of dietary fibres.

The product can be characterized in that the fat comprises the essential fatty acids with an omega-3 to omega-6 fatty acid ratio of 1:0.5 to 1:3.0, more preferably 1:0.8 to 1:2.0, and most preferably 1:1.0 to 1:1.3.

The product can be characterized in that the fatty acids comprise 25-40%, saturated fatty acids, 30-45%, monounsaturated fatty acids, and 15-35%, di- and tri-unsaturated fatty acids.

The product can be characterized in that the levels of docosahexaenoic acid (DHA) and eicosapentaenoic acid (EPA) are optimised and constitute between 1.9-2.3%, preferably about 2.1% of the total fat.

The product can be characterized in that the whole egg content in the food composition product is supplied from especially fed chicken in order to give the whole egg product the preferred balance between saturated, monounsaturated and polyunsaturated fat, and the balance between omega-3 and omega-6 fatty acids in a rating of 1:0.7-1.5.

The product can be characterized in that it comprises all 20 amino acids including the 8 essential amino acids.

The product can be characterized in that it comprises vitamins A, B, C, D, and E, and the essential minerals iron (Fe), zinc (Zn), calcium (Ca), phosphorus (P), magnesium (MG) copper (Cu), manganese (Mn), chromium (CR), selenium (Se), silica (Si), potassium (K), and sodium (Na), wherein potassium is present in high amounts, and sodium in small amounts, in physiological doses.

The product can be characterized in that it is in the ph-range of 5.8 to 6.2 and acts like a buffer.

The product can be characterized in that it is gluten free.

The product can be characterized in that it comprises no, artificial sweeteners, preservatives, flavorings, dyes or thickening agents.

The product can be characterized in that the food composition product contains less than 75 kCal per portion serving, and correspondingly less than 425 kCal per 100 g powdermix of the dry food composition product.

The product can be packaged as a single portion pack, a liquid, or a bar or in such other form as may be desired.

An aspect of the invention provides an ingestible composition comprising a plurality of components consisting of fat, carbohydrates, proteins, vitamins and minerals, wherein relative amounts of said components of said composition are chosen such that an intake of said product reduces the effects of metabolic syndrome.

The product weight ratio between carbohydrates, proteins and fat can be of the magnitude of 0.8-2.0:1:0.1-0.4, respectively, wherein the carbohydrates are chosen from the group of foodstuffs having a low glycemic index.

The product weight ratio between carbohydrates, proteins and fat can be of the magnitude of 0.8-2.0:1:0.1-0.3, respectively, wherein the carbohydrates are chosen from the group of foodstuffs having a low glycemic index.

The carbohydrates can be derived from leguminous plants, preferably yellow peas, and rosaceous plants, preferably apples and rose hips.

At least 15% of the overall content is derived from yellow peas, at least 10% from apples, and at least 5% from rose hips.

An overall meal of the product can have a GL-value below about 20, or below about 10.

The carbohydrates can be in the form of simple sugars from the group consisting of glucose, saccharose, fructose, maltose and lactose.

The sugars can be present in a content of 2.1-2.5%, preferably about 2.3%, glucose, 1.8-2.2%, preferably about 2.0%, saccharose, 4.0-4.8%, preferably about 4.4% fructose, <0.03-0.05%, preferably about <0.04%, maltose and 12.9-15.7%, preferably about 14.3% lactose.

A ready-to-drink serving of said ingestible composition can be either a low lactose content or alternatively is completely free from lactose.

The product can comprise 7-12% of dietary fibres.

The fat in the product can comprise the essential fatty acids with an omega-3 to omega-6 fatty acid ratio of 1:0.5 to 1:3.0, more preferably 1:0.8 to 1:2.0, and most preferably 1:1.0 to 1:1.3.

The fatty acids can comprise 25-40%, saturated fatty acids, 30-45%, monounsaturated fatty acids, and 15-35%, di- and tri-unsaturated fatty acids.

The levels of docosahexaenoic acid (DHA) and eicosapentaenoic acid (EPA) can be optimised and constitute between 1.9-2.3%, preferably about 2.1% of the total fat.

The whole egg content in the composition can be supplied from especially fed chicken in order to give the whole egg product the preferred balance between saturated, monounsaturated and polyunsaturated fat, and the balance between omega-3 and omega-6 fatty acids in a rating of 1:0.7-1.5.

The product can comprise 20 amino acids including the 8 essential amino acids.

The product can comprise vitamins A, B, C, D, and E, and the essential minerals iron (Fe), zinc (Zn), calcium (Ca), phosphorus (P), magnesium (MG), copper (Cu), manganese (Mn), chromium (CR), selenium (Se), silica (Si), potassium (K), and sodium (Na), wherein potassium is present in high amounts, and sodium in small amounts, in physiological doses.

The product can have a Ph range of about 5.8 to about 6.2 and act like a buffer.

The product can be gluten free.

The product can be produced to have no artificial sweeteners, preservatives, flavorings, dyes or thickening agents.

The product can contain less than 75 kCal per portion serving, and correspondingly less than 425 kcal per 100 g in a powder mix dry form.

An aspect of the invention provides a method of treating metabolic syndrome comprising the step of consuming each day between about fifteen grams and about eighty grams of an ingestible composition comprising fat, carbohydrates, proteins, vitamins and minerals, wherein relative amounts of said components of said composition are chosen such that an intake of said product reduces the effects of metabolic syndrome.

An aspect of the invention provides an ingestible composition for use in treating metabolic syndrome comprising a plurality of components consisting of fat, carbohydrates, proteins, vitamins and minerals, wherein relative amounts of said components of said composition are chosen such that an intake of said product reduces the effects of metabolic syndrome.

An aspect of the invention provides an ingestible composition comprising a plurality of components consisting of fat, carbohydrates, proteins, vitamins and minerals, wherein relative amounts of said components of said food composition product are chosen such that an intake of said product reduces the effects of metabolic syndrome, said composition used in the preparation of a medicament for the treatment of at least one of metabolic syndrome, diabetes, thrombosis, and obesity.

An aspect of the invention comprises a method of treating at least one of metabolic syndrome, diabetes, and obesity comprising the step of consuming each day for at least a period of about two weeks between about fifteen grams and about eighty grams of an ingestible composition comprising: pea protein, whole egg powder, whey powder, dried apples, rose hip powder, egg albumin powder, ground sugar beet powder, milk protein, fructose, ascorbic acid.

A method of reducing thrombotic risk comprising the step of consuming each day for at least a period of about two weeks between about fifteen grams and about eighty grams of an ingestible composition comprising: pea protein, whole egg powder, whey powder, dried apples, rose hip powder, egg albumin powder, ground sugar beet powder, milk protein, fructose, ascorbic acid.

An aspect of the invention provides an ingestible composition for use in treating at least one of metabolic syndrome, diabetes, and obesity comprising: pea protein, whole egg powder, whey powder, dried apples, rose hip powder, egg albumin powder, ground sugar beet powder, milk protein, fructose, ascorbic acid.

An aspect of the invention provides an ingestible composition for reducing thrombotic risk comprising the step of consuming each day for at least a period of about two weeks between about fifteen grams and about eighty grams of an ingestible composition comprising: pea protein, whole egg powder, whey powder, dried apples, rose hip powder, egg albumin powder, ground sugar beet powder, milk protein, fructose, ascorbic acid.

Aspects of the invention can lower or ultimately normalise a person's blood glucose level and reduce blood glucose peaks by means of the chosen food ingredients and the nutritional balance of the ingestible composition. Combined with a relatively low content of slow carbohydrates, the composition gives substantially complete nutrition with very little energy content. The composition can act as both a short term and a long term stabilizer to the blood glucose response. The blood glucose balancing effect can be measured as a Glycemic Index, according to international standards, and can be considered as having a rate of not more than about GI=25. This composition can improve the metabolism of the body and have a long-term effect in better health, and can reduce one or more of the parameters included within the diagnosis of the metabolic syndrome, or Syndrome-X.

The composition can improve glucose tolerance by reducing the glucose levels going into the cells, which in turn will lower the glucose load in the cells, and this will improve the ability of the cells to easier adopt the glucose and ultimately reducing the need of to high glucose levels, that are within the diagnosis of the metabolic syndrome, in order to give enough energy to the cells.

The composition can balance insulin excretion and improve the sensitivity of the insulin receptors in the muscle and fat cells. As known to those of skill in the art, this can be beneficial where an individual has an increased BMI, and especially high central abroid obesity, there can be too many insulin receptors within the fat cells, resulting in high insulin fasting levels and accordingly also an exhaustion of the Pancreas insulin production, and accordingly create a risk of either limiting further insulin production and/or set down the efficiency of the insulin receptors, both being within the diagnosis of metabolic syndrome.

The composition can, when used in accordance with the teachings herein, in certain circumstances, reduce and/or obviate the need for medical therapy with additional insulin in Diabetes type 1 patients, which may be within the diagnoses of the metabolic syndrome.

The composition can, when used in accordance with the teachings herein, in certain circumstances, lower and/or obviate the need for medical therapy with anti glucose pharmaceuticals and/or additional insulin and ultimately the effects may cure the Diabetes type 2, which is within the diagnoses of the metabolic syndrome.

The composition can, when used in accordance with the teachings herein, in certain circumstances, improve blood cholesterol values by having a positive effect on HDL cholesterol and by lowering the total cholesterol level thus improving the ratings between the HDL and LDL cholesterol and ultimately normalise the cholesterol balance and total levels.

The composition can, when used in accordance with the teachings herein, in certain circumstances, lower the rate of Triglycerides.

The composition can, when used in accordance with the teachings herein, in certain circumstances, improve blood pressure and especially lower the diastolic pressure, by reducing the triglycerides and/or balancing the HDL/LDL rating and the total cholesterol the this has in turn positive effects on blood pressure.

The composition can, when used in accordance with the teachings herein, in certain circumstances, lower central (android) obesity especially for those persons who have a BMI index over about 25, or in general those where a high intake of glucose through food having a high carbohydrate content can trigger the insulin hormone to start transforming glucose molecules into fatty molecules and after filling up the glycogene depots in the liver and muscles and generate the insulin hormone to store additional energy in terms of fat. Android obesity is a high health risk factor and as a reference you may use the ‘measurement around the stomach’ and if this is over 1020 mm for a male and 970 mm for a female this person is within the diagnoses of the metabolic syndrome.

The composition can, when used in accordance with the teachings herein, in certain circumstances, lower BMI (body mass index) significantly for those persons having increased body fat ratio. Using the effects of the invention will reduce the fat storage within the body and thus the BMI measurement. A person with a BMI rating over 25 is within the diagnoses of the metabolic syndrome.

The composition can, when used in accordance with the teachings herein, in certain circumstances, decrease the plasminogen activator inhibitor and thus lowering the hyperuricemia by lowering the uric acid in the blood system, which is within the diagnoses of the metabolic syndrome.

### BRIEF DESCRIPTION OF THE DRAWINGS

FIGS. 1A and 1B are a graph of table results from performing a first study using the ingestible composition of the present invention for metabolic syndrome;

FIGS. 2A and 2B are a second table of test results from performing a first study using the ingestible composition of the present invention for metabolic syndrome;

FIG. 3 is a graph of test results from performing a second study using the ingestible composition of the present invention for metabolic syndrome;

FIG. 4 is a graph of test results from performing a second study using the ingestible composition of the present invention for metabolic syndrome;

FIG. 5 is a graph of test results from performing a second study using the ingestible composition of the present invention for metabolic syndrome;

FIG. 6 is a graph of test results from performing a second study using the ingestible composition of the present invention for metabolic syndrome;

FIG. 7 is a graph of test results from performing a second study using the ingestible composition of the present invention for metabolic syndrome;

FIG. 8 is a graph of test results from performing a third study using the ingestible composition of the present invention for metabolic syndrome; and,

FIGS. 9A and 9B are a graph of test results from performing a third study using the ingestible composition of the present invention for metabolic syndrome.

### DETAILED DESCRIPTION OF THE INVENTION

An embodiment of the invention is an ingestible composition comprised of a combination of plants, fruits, vegetables, fibre, whole egg or parts thereof, milk proteins, cheese fats and cheese proteins. According to the present invention an ingestible composition (also referred to as a food composition product or food supplement) is provided which product can replace all or part of at least one entire meal. Furthermore, the ingestible composition substantially eliminates the disadvantages associated with previously known ingestible compositions.

A dry ingestible composition for mixing with a drinkable liquid according to the invention is distinguished primarily by the fact that the ingestible composition comprises all the essential food components readily accessible, wherein the relative amounts of the components fat, carbohydrates, proteins, vitamins and minerals of said ingestible composition are chosen such that an intake of said ingestible composition provides the consumer with a stable blood glucose level. The ingestible composition preferably has a weight ratio between carbohydrates, proteins and fat of the magnitude of 0.8-2.0:1:0.1-0.4, and more preferably a weight ratio of the magnitude of 0.8-2.0:1:0.1-0.3, respectively, wherein the carbohydrates are chosen from the group of foodstuffs having a low glycemic index. The weight ratio between carbohydrates, proteins and fat is yet more preferably 0.8-1.5:1:0.1-0.3, and most preferably 0.7-1.3:1:0.1-0.3. Further weight ratios between carbohydrates, proteins and fat could be 1.2-1.8:1:0.1-0.3, 0.8-2.0:1:0.15-0.25, 1.0-1.9:1:0.15-0.25. Said fat, carbohydrates, proteins, vitamins and minerals are derived from a diversity in food groups including fruit, vegetable, plant, dairy, egg and other protein sources, and colloidal water sources, resulting in a physiologically balanced ingestible composition that can to replace all or part of an entire meal.

The “glycemic index” (GI) is a measure of the degree to which the concentration of glucose in the blood rises after consumption of certain foodstuffs. A low glycemic index (GI) here refers to foodstuffs with a GI-value between 0-60. The glycemic index (GI) may be calculated using two different references, that is to say either the reference white bread or the reference glucose. In the measurements made on the ingestible composition according to the invention, the reference glucose was utilised. In order to estimate the overall glycemic effect of a meal, the concept of “glycemic load” (GL) (GI× dietary carbohydrate content) has been introduced. As the GI compares corresponding amounts of carbohydrates, providing a measure of carbohydrate quantity, but not quality, the GL-VALUE provides the glycemic effect of realistic portion sizes of different foods. The GL-VALUE is similar to the GI-VALUE, a measure of the rise of the blood glucose and the subsequent secretion of insulin in the blood stream, but including the aspect of the amount of carbohydrates available in a portion of food (see Foster-Powell K., Holt S H., Brand-Miller J C., Am. J. CLIN. NUTR. 76:5-56,2002), the contents of which are incorporated herein by reference.

According to one preferred embodiment of the invention, the carbohydrates are derived from leguminous plants, preferably yellow peas, and rosaceous plants, preferably apples and rose hips, wherein said leguminous plants and rosaceous plants contribute in giving the ingestible composition its advantageously low GL-VALUE. However, carbohydrates may, of course within the scope of the invention, be chosen from other plants having a low glycemic index. Apples and rose hips may furthermore be substituted with pears, peaches, plums, or the like, all of which being rosaceous plants with a low glycemic index. In a further preferred embodiment of the invention, at least 15% of the overall content of the ingestible composition is derived from yellow peas, at least 10% from apples and at least 5% from rose hips. The content of the mentioned ingredients preferably does not exceed 30% for yellow peas, 25% for apples, and 15% for rose hips so as to maintain the important balance between carbohydrates, proteins and fat. A study performed by the present inventors on a ingestible composition including vitaceous plants has shown that such an inclusion significantly increases the GL-value for the overall ingestible composition.

In addition, said composition is also proven to have a satiating effect for a longer period of time after a consumed meal, which effect is of great importance so as to keep feelings of hunger away. As has been disclosed in a previous study (Holt S H., Miller J. C., Petocz., Farmakalidis E., Eur. J. Clin. Nutr. 49 (9):675-690, 1995), different foods differ greatly in their satiating capacities. The ingestible composition according to the invention provides said satiating effect due to a number of reasons mentioned in the study above. The low degree of fat in the ingestible composition, is proven advantageous, as fatty foods are shown not to be satisfying, as well as the inclusion of foodstuffs such as eggs and apples, both of which having a high satiety index, as defined in said article. Furthermore, the ingestible composition according to the invention has a balanced ratio between certain important components such as the contents of protein, fibre, and water. Said components are shown in the above mentioned study to have a positive effect on said satiety.

The mutual relation between carbohydrates, proteins and fat in the ingestible composition has surprisingly been proven to be of significant value in maintaining the overall body system in balance, that is, avoiding deficiencies and excesses of nutrients in the systems of the body. When the body is in balance, intake of nutrients and energy is sufficient to maintain tissue needs and the amounts of nutrients and energy entering and exiting the body are equal. The above mentioned relation further provides the consumer with an amount of each component corresponding to an adequate meal when being provided with a portion of the ingestible composition according to the invention. In one embodiment of the invention the carbohydrates, proteins and fat are advantageously present in the form of 10-20% whole eggs, 4-15% egg albumen, 10-25% whey, 15-30% yellow pea, 10-25% apple, and 5-15% rose hips.

An aspect of the invention provides an ingestible composition as described herein, which is balanced so that the overall meal has a GL-value below 20, preferably below 10. As earlier mentioned, carbohydrates are metabolised into glucose by the digestive system of the human body. Until the mid-eighties, it was generally considered that the size of the carbohydrates was of primary importance regarding the blood glucose response. Today, it is known that the same carbohydrate gives rise to different blood glucose responses, due to the form in which it is included in the foodstuffs (see e.g., BJORCK I., LILJEBERG H., GRANFELDT Y, Akerberg A., Scand. J. NUTR./NäRINGSFORSKNING VOL. 40:38-42, 1996). Recently, the “GLYCEMIC index” (GI) has replaced the terminology of “fast” and “slow” carbohydrates which were related to the size of the sugars included. High GI-values indicate a rapid increase in blood glucose, and low GI-values indicate a delayed absorption rate for the glucose; i.e., carbohydrates with low GI-values are more slowly digested and absorbed. As the level of blood glucose is kept at a more even level, feelings of hunger are kept away for a prolonged period also avoiding unnecessary and unhealthy cravings for foodstuffs rich in carbohydrates. Thus, said problems are avoided with the ingestible composition having said GL-value, which GL-value is a product of a low GI-value and the amount of available carbohydrates. However, in addition to the ingestible composition having a low GL-value, the satiating effect of said product is of equal importance in order to avoid feelings of hunger.

However, even though carbohydrates from different sources may be associated with individual GI-VALUES, the GI-value of each of the carbohydrates put together does not determine the GI of the foodstuffs. The GI is influenced by a number of factors, for example, the biochemical structure of the carbohydrate, a high amylose/amylopectin ratio, a high degree of native starch, presence of anti-nutritional substances with the ability of inhibiting amylose, and the co-ingestion of fat, fibre and protein. A problem thus occurs when trying to compose a meal which is to provide the consumer with a low glycemic index for the overall composition. Due to the contribution of a number of factors to the glycemic index, a meal properly balanced so as to avoid fluctuations in the blood glucose level may be difficult to compose.

One important factor in obtaining a low GL ingestible composition is, as mentioned, the co-ingestion of further components. As will be shown in Example 2, the ingestible composition according to the invention exhibits a desired glucose absorption rate when having a weight ratio of the carbohydrates, proteins and fat as described above.

Furthermore, the choice of carbohydrate source is, as also mentioned, another important factor in obtaining a low GL ingestible composition. Accordingly, carbohydrates having low GI-Values are chosen. According to one embodiment of the invention the ingestible composition has a content comprising yellow peas, apples, and rose hips as described above, all of which comprise carbohydrates with low GI-VALUES. The present inventors have found that when foodstuffs with an average GI-VALUE, that is between 60-90, are included, the overall ingestible composition obtains a significantly higher GL-VALUE when compared to a composition comprising only carbohydrates with a low GI-VALUE.

According to a further embodiment of the invention, the carbohydrates, when decomposed provide the human body system with simple sugars from the group consisting of glucose, saccharose, fructose, maltose and lactose. The different sugars are advantageously present in a percentage of 2.1-2.5%, preferably about 2.3%, GLUCOSE, 1.8-2.2%, preferably about 2.0%, saccharose, 4.0-4.8%, preferably about 4.4% fructose, <0.03-0.05%, preferably about <0.04%, maltose, and 12.9-15.7%, preferably about 14.3% lactose.

However, it is also possible to alter the contents of the different types of sugar within the scope of the invention. In one embodiment according to the invention the ingestible composition is low lactose or completely free from lactose in order to meet the demands for such products. A low lactose product is directed to people who cannot tolerate milk, and thus lactose, in small amounts. It is important to note that the ready to drink product-when one serving of the ingestible composition consisting of 18 g powder is mixed with at least 20 cl water-gives less than 1% lactose content per serving, which is to be recognized as low lactose content.

According to one embodiment of the invention, the ingestible composition comprises physiological doses of dietary fibres, i.e., between 7-12%, preferably about 8.4%. The GL of a meal is affected by the content of fibres, as a high degree of fibres helps to lower the glycemic index. Fibres are not digested, but are however necessary in order for the bowels to function correctly. The high content of fibres in the ingestible composition according to the invention is advantageous in improving motility (bowel movements) and thus enhancing the consumer's ability to process food efficiently. In addition, the fibres help the elimination of toxins in the cells and furthermore is recognized to reduce the cholesterol content in the blood system.

In a presently preferred embodiment, the fat of the ingestible composition comprises the essential fatty acids with an omega-3 to omega-6 fatty acid ratio of 1:0.5 to 1:3.0, more preferably 1:0.8 to 1:2.0, and most preferably 1:1.0 to 1:1.3. A correct balance is significant in order to maintain normal cellular and other functions. As a typical western diet consists of far more omega-6 fatty acids than omega-3 fatty acids, many meals are lacking the essential amount of the desired fatty acids and have moreover an inadequate balance of said fatty acids. The fat of the product is further composed to meet the needs for saturated, and mono-, di-, tri- and polyunsaturated fatty acids. The fat preferably comprises preferably 25-40%, more preferably 28-35%, most preferably about 29-33%, saturated fatty acids, preferably 30-45%, more preferably 32-39%, most preferably about 33-37%, monounsaturated fatty acids, and preferably 15-35%, more preferably 25-35%, most preferably about 29-33%, di- and tri-unsaturated fatty acids. Polyunsaturated fatty acids are preferably present in an amount of 3.0-3.6%. The balance between the three main groups of fatty acids is thus optimised. The content of fat in a properly balanced meal is essential or at least generally desirable; fats contain important fat soluble vitamins and essential fatty acids which cannot be produced by the human body. The fatty acids are preferably chosen from the group consisting of myristic acid, palmitic acid, palmitoleic acid, heptadecanoic acid, stearic acid, oleic acid, linoleic acid, alfa-linolenic acid, arachidic acid, eicosadienoic acid, behenic acid, docosahexaenoic acid (DHA) and eicosapentaenoic acid (EPA). However, the invention is not restricted to the fatty acids mentioned herein. Other fatty acids which are within the scope of the invention may also be chosen. In addition, the levels of docosahexaenoic acid (DHA) and eicosapentaenoic acid (EPA) are optimised and constitute between about 1.9-2.3%, preferably about 2.1% of the total fat. Cholesterol is also present in amounts which meet the body's needs.

Preferably, the fat of the ingestible composition is mainly derived from whole eggs. To achieve the preferred balance between saturated, monounsaturated and polyunsaturated fat the whole egg content in the ingestible composition is supplied from especially fed chicken in order to give the whole egg product a balance between omega-3 and omega-6 fatty acids in a rating of 1:0.7-1.5.

The ingestible composition is balanced with ingredients so that it offers a complete meal with less than 75 Kcal per portion serving, more preferably less than 70 Kcal and most preferably less than 68 Kcal and correspondingly less than 425 Kcal more preferably less than 400 Kcal and most preferably less than 385 kcal per 100 g powdermix of the dry ingestible composition.

The term serving here refers to a portion of the dry ingestible composition when dispersed in a drinkable liquid. The ingestible composition is furthermore an excellent source of protein (30-45 g protein, more preferably 35-40 g, and most preferably 37-39 g per 100 g dry food composition powder), thus containing all 20 amino acids including the 8 recognized essential amino acids in physiological doses. The amino acids are suitably derived from sources such as whey, yellow pea protein, egg albumen, and whole eggs. The ingestible composition according to the invention is designed to ensure that a daily allowance of proteins, and essential amino acids, is met. The content of protein is further designed so that the composition of the ingestible composition provides a positive nitrogen balance which promotes water excretion, visceral fat utilisation and lean body mass preservation.

An adequate balance of vitamins is needed in order to convert food into energy, maintain the body, and keep it functional. Therefore, the ingestible composition comprises vitamins A, B, C, D, and E in physiological doses, wherein vitamins C and E contribute as important antioxidants.

In addition, the ingestible composition according to the invention comprises essential minerals and trace elements in physiological doses; iron (Fe), zinc (Zn), calcium (Ca), phosphorus (P), magnesium (Mg), copper (Cu), manganese (Mn), chromium (Cr), selenium (Se), silica (Si), potassium (K), and sodium (Na), wherein potassium is present in high amounts, and sodium in small amounts. Minerals are essential for the metabolic processes in the body. They act as catalysts for the major body processes, wherein they act in an interrelated manner. A lack of minerals in the body can create a deficiency, and excess may create disturbances in the body process, therefore the amounts and balance between the minerals is of great importance.

Said product is in the pH-range of 5.8 to 6.2 and acts like a buffer, thus having a positive effect on people suffering from G. E. R. D. (gastroesophageal reflux disease) and Non Ulcer Dyspepsia.

In order to meet the demands of people with gluten intolerance, the ingestible composition is made gluten free.

Because of the demand for a balanced meal which is ready to use, the ingestible composition is preferably prepaced in a dry powder form in single portion packs with a net dry weight of preferably 15-20 g, more preferably 17-19 g, and most preferably about 18 g. The dry powder is readily dissolved in a drinkable liquid, preferably water, and may be consumed promptly. Missing out on meals such as breakfast, or eating fast food or snacks, due to a tight schedule may thus be avoided, and a healthy lifestyle can be maintained. In order to replace all or part of a breakfast, one or two single portion packs are preferably used, depending on the individuals need. Children are suitably provided with one single portion pack for breakfast, while adults suitably are required to consume one to two single portion packs. In order to obtain a proper intake of nourishment equivalent with a dinner, males should consume up to four single portion packs, while women are required to consume up to three. The consumption of said product should, however, not exceed more than an equalling number of portion packs containing ca 7 g of protein each corresponding to a daily intake of 0.75 g protein per kilogram body weight.

The dry powder has proven to be advantageously stable to degradation, especially with regard to fatty acids, for at least three months in [25° C.] and [40° C.,] respectively. The prepacking of the ingestible composition also prevents exposure to air and moisture, and subsequent oxidation, when compared to packages where the consumer serves out a portion from a larger quantity of dry powder.

Said product may also be mixed with a drinkable liquid, such as milk, juice or the like. When the ingestible composition is mixed with juice, the content of carbohydrates is increased, providing consumers with greater needs for carbohydrates, such as growing children, to have a meal with higher energy content. A weight ratio between carbohydrates, proteins and fat is in this case preferably 1.0-2.0:1:0.1-0.3, more preferably 1.2-1.8:1:0.1-0.3, and most preferably 1.4-1.6:1:0.1-0.3.

Furthermore, the ingestible composition may also be produced in the form of a bar, thus providing the consumer with one or more portions of the ingestible composition in an expediently packaged form, which is/are easy to consume. The bar may be produced by any method known in the art.

The ingestible composition may be used as a sole source of nutrition of the daily food consumption, or may be used as a replacement for all or part of one or more meals throughout the day. The ingestible composition according to the invention supplies the consumer with an inexpensive meal which can lower the expenses for foodstuffs and additional food supplements.

The nutritive substances of the ingestible composition according to the invention are derived from both animal and vegetable sources, thereby providing all the amino acids, including the essential amino acids, fatty acids, and slow-working carbohydrates in a proper balance, all of which contribute to a low glycemic index. No artificial sweeteners, preservatives, flavorings, dyes or thickening agents are added to the product.

Moreover, the ingestible composition is advantageous in that it acts like an insulin mimetic. Said product has also proved to provide a stable blood glucose level, to prevent insulin spikes. In addition, basal insulin levels are lowered in all people, and long acting (basal insulin level) and short acting (insulin spike) insulin requirements are significantly reduced with insulin dependent diabetics.

The invention will now be described in greater detail with reference to the following examples, of which Example 1 describes the ingredients of one ingestible composition according to the invention, and Example 2 illustrates the effect on blood glucose level after consumption of a ingestible composition according to the invention.

### EXAMPLE 1

The ingredients of the ingestible composition according to one embodiment of the invention, wherein the components are given in grams per 100 grams or grams per kg of the dry ingestible composition. The composition of the fatty acids are given in percentage value.

| | | g/100 g | g/18 g port. |
|---|---|---|---|
| Protein N*6.25 | 38.3 g/100 g | 38.3 | 6.89 |
| Moisture | 5.9 g/100 g | 5.9 | 1.06 |
| Ash | 4.8 g/100 g | 4.8 | 0.86 |
| Fat | 9.1 g/100 g | 9.1 | 1.64 |
| Carbohydrates calculated | 33.5 g/100 g | 33.5 | 6.03 |
| Dietary Fibers | 8.4 g/100 g | 8.4 | 1.51 |
| | | 100.0 | 18.00 |
| Energy kJ | 1557.3 kJ/100 g | 1557 | 280 |
| Energy kcal | 372.2 kcal/100 g | 372 | 67 |
| Carbohydrates composition | | | |
| Glucose | 2.3 g/100 g | 2.3 | 0.41 |
| Saccharose | 2.0 g/100 g | 2.0 | 0.36 |
| Fructose | 4.4 g/100 g | 4.4 | 0.79 |
| Maltose | <0.04 g/100 g | <0.04 | <0.0072 |
| Lactose | 14.3 g/100 g | 14.3 | 2.57 |
| Total calculated | 33.5 | 33.5 | 6.03 |
| carbohydrates | | | |
| Amino Acids composition | | | |
| Tryptophan (BCR) | 4.2 g/kg | 0.4 | 0.08 |
| Cystine | 6.4 g/kg | 0.6 | 0.12 |
| Methionine | 7.0 g/kg | 0.7 | 0.13 |
| Aspartic Acid | 42.09 g/kg | 4.3 | 0.77 |
| Threonine | 16.0 g/kg | 1.6 | 0.29 |
| Serine | 21.6 g/kg | 2.2 | 0.39 |
| Glutamic Acid | 59.8 g/kg | 6.0 | 1.08 |
| Proline | 15.8 g/kg | 1.6 | 0.28 |
| Glycine | 14.6 g/kg | 1.5 | 0.26 |
| Alanine | 18.2 g/kg | 1.8 | 0.33 |
| Valine | 22.7 g/kg | 2.3 | 0.41 |
| Isoleucine | 19.9 g/kg | 2.0 | 0.36 |
| Leucine | 30.9 g/kg | 3.1 | 0.56 |
| Tyrosine | 13.2 g/kg | 1.3 | 0.24 |
| Phenylanilline | 19.4 g/kg | 1.9 | 0.35 |
| Histidine | 9.8 g/kg | 1.0 | 0.18 |
| Omithine | <0.1 g/kg | <0.01 | <0.0018 |
| Lysine | 27.1 g/kg | 2.7 | 0.49 |
| Arginine | 27.4 g/kg | 2.7 | 0.49 |
| Hydroxyproline | <0.1 g/kg | <0.01 | <0.0018 |
| Sum | 376.9 g/kg | 37.7 | 6.78 |
| Fatty Acid composition | % Tot 9.1 g/100 g | | |
| Myristic Acid C14 | 0.5 | 0.05 | 0.008 |
| Palmitic Acid C16 | 21.7 | 1.97 | 0.355 |
| Palmitoleic Acid C16: 1 | 2.7 | 0.25 | 0.044 |
| (mono unsat) | | | |
| Heptadecanoic acid C17 | 0.2 | 0.02 | 0.003 |
| Stearic Acid C18 | 8.5 | 0.77 | 0.139 |
| Oleic Acid C18: 1 | 32.1 | 2.92 | 0.526 |
| (mono unsat) | | | |
| Omega 3/6 (di, tri, and | | | |
| tetra-unsatur) | | | |
| Linolic Acid C18: 2-6 | 18.1 | 1.65 | 0.296 |
| Alfa-Linoleic Acid C18: | 12.5 | 1.14 | 0.205 |
| 3-3 | | | |
| Eicosadienoic Acid C20: | 0.9 | 0.08 | 0.015 |
| 4-6 | | | |
| EPA C20: 5-3 | 0.3 | 0.03 | 0.005 |
| Behenic Acid C22: 5-3 | 0.3 | 0.03 | 0.005 |
| DHA C22: 6-3 | 1.8 | 0.16 | 0.029 |
| Total Fatty Acids | 99.6 | 9.06 | 1.631 |
| Cholesterol | 260 mg/100 g | 0.26 | 0.05 |
| Ammonia | 3.7 g/kg | 0.37 | 0.07 |
| Minerals | | mg/100 g | mg/18 g |
| Sodium Na | 570 mg/100 g | 0.6 | 0.10 |
| Iron Fe | 5 mg/100 g | 5.0 | 0.90 |
| Zinc Zn | 1.9 mg/100 g | 1.9 | 0.34 |
| Calcium Ca | 250 mg/100 g | 250.0 | 45.00 |
| Phophorus P | 470 mg/100 g | 470.0 | 84.60 |
| Magnesium Mg | 110 mg/100 g | 110.0 | 19.80 |
| Potassium K | 870 mg/100 g | 870.0 | 156.60 |
| Copper Cu | 0.25 mg/100 g | 0.3 | 0.05 |
| Manganese Mn | 1.6 mg/100 g | 1.6 | 0.29 |
| Chromium Cr | 0.07 mg/kg | 0.7 | 0.13 |
| Selenium Se | 0.14 mg/kg | 1.4 | 0.25 |
| Silica Si | 14000 mg/100 g | 14000.0 | 2520.00 |
| Retinol, Vitamin A | 3300 IE/100 g | 3300 | 594 |
| Vitamin D3 | <1 ug/100 g | <0.001 | <0.0002 |
| dl-Alfa-Tocopherol, | 8 mg/100 g | 8.0 | 1.44 |
| Vitamin E | | | |
| Thiamine Hydrochloride | 0.14 mg/100 g | 0.1 | 0.03 |
| B1-HC1 | | | |
| Niacine Acid B3 | 0.6 mg/100 g | 0.6 | 0.11 |
| Pyridoxine HCl, Vitamin | 0.2 mg/100 g | 0.2 | 0.04 |
| B6 | | | |
| Folic Acid Bc/M | 72 ug/100 g | 0.1 | 0.01 |
| Cyanocobalamin | 1.2 ug/100 g | 0.001 | 0.0002 |
| Beta Carotene | 0.61 mg/100 g | 0.6 | 0.11 |
| Ascorbic Acid, Vitamin C | 100 mg/100 g | 100.0 | 18.00 |

Each portion of the ingestible composition (18 grams) supplies the consumer with about 7 grams protein, about 6 grams carbohydrates, about 1.6 grams of fat, and with 67 kcal.

### EXAMPLE 2

The ingestible composition according to Example 1 was analysed with regard to B-glucose (Hemocue, Angelholm), S-insulin (Klinisk Kemiska Laboratoriet, MAS), and P-glucose (Klinisk Kemiska Laboratoriet, Universitetssjukhuset in Lund). Measurements were similarly made on the reference glucose, and on a ingestible composition having an altered composition of carbohydrates. The same test person, a male, having a body weight of 80 kg was tested on four different occasions. Prior to each test a fasting glucose value was obtained. Measurements were then made 30,60, 90, and 120 minutes after consumption of said composition. The glycemic index was calculated for a meal consisting of 158 grams of the ingestible composition dispersed in about 8 dl of water, which amount corresponds to about four portions of said composition, and for 158 grams of the ingestible composition dispersed in about 6 dl of water. The data was graphically represented with the glucose concentration on the y-axis, and with time on the x-axis. The area under the blood glucose curve was then calculated. The same procedure was applied to the reference, consisting of 50 grams of glucose dispersed in 3 dl of water. The glycemic index was given as percentage value when the areas under each blood glucose curve were being compared.

The result of the present study of a ingestible composition according to the invention was a calculated GL-value of 4 and 8, respectively. Such a GL-value implies that consumption of said amount of the composition according to the invention provokes an increase of blood glucose 4% and 8%, respectively, as great as that for consumption of an amount of glucose equivalent to that of said product. This low GL-value illustrates the positive effect of the present invention on the blood glucose level. Similarly, as the blood glucose curve was shown to have a flattened appearance, the ingestible composition is proved to have a controlled and delayed absorption rate of the digested carbohydrates. The levels of S-insulin were also exhibiting low values throughout the study.

A comparative study was made on a ingestible composition comprising carbohydrates with an average GI-Value taken from the group consisting of vitaceous plants, specifically grapes which have a GI-Value of 61. This study was performed generally with the same ingestible composition but with added grapes, totally approximately 10%, and with corresponding decrease of rose hip and yellow peas, and measurements were made as above. The result of the study was a GL-value of 24.4, a value significantly higher than the ingestible composition according to the invention. This elevated value indicates a more rapid absorption of glucose and thus the more unfavourable absorption pattern described above. The elevated B-glucose and P-glucose peaks were followed by unfavourable S-insulin spikes, also illustrating said unfavourable absorption pattern.

The data obtained in the above mentioned measurements are given in Table 1. The values for B-glucose and P-glucose are in mmol/l, and the values for S-insulin are in mlE/l.

TABLE 1

| | | | B-glu | S-Ins | B-glu | S-Ins | P-glu | B-glu | S-Ins | P-glu |
|---|---|---|---|---|---|---|---|---|---|---|
| Time | B-glu (I)¹ | P-glu (I) | (II)² | (II) | (glu)³ | (glu) | (glu) | (alt)⁴ | (alt) | (alt) |
| 0 | 5.4 | 4.6 | 4.8 | 4 | 4.7 | 4 | 5.6 | 4.5 | 4 | 5.2 |
| 30 | 5.9 | 4.2 | 5.5 | 30 | 10.4 | 28 | 11.2 | 6.8 | 41 | 7.8 |
| 60 | 3.8 | 3.4 | 4.6 | 12 | 6.9 | 41 | 7.9 | 3.3 | 18 | 3.9 |
| 90 | 4 | 3.6 | 4.1 | 6 | 4.6 | 19 | 5.4 | 2.8 | 4 | 3.5 |
| 120 | 5.1 | 3.6 | 4.4 | 6 | 2.4 | 3 | 3.3 | 4.1 | 3 | 4.7 |

¹(I) A first test on 158 g of a food composition product according to the invention.
²(II) A second test on 158 g of a food composition product according to the invention.
³(glu) A test on 50 g of the reference glucose.
⁴(alt) A test on 50 g of an altered food composition product according to the invention.

Further measurements performed on a ingestible composition according to the invention as described in Example 1 surprisingly disclosed that an additional intake of said ingestible composition caused a lesser raise in the GL-value than would have been expected from the intake the amount of carbohydrates contained in said product. That is to say, normally an added intake of a certain amount of carbohydrates shows a corresponding rise in the blood glucose, proportional to that amount. However, the ingestible composition according to the invention advantageously showed a lesser rise in the blood glucose when compared to the additional intake of that certain amount of ingestible composition. Without being in any way limited by the following theoretical explanation of why further consumption of additional amounts of said ingestible composition exhibits a rather stable level of blood glucose both after a first intake of an amount of said product and after a second intake of an amount of said product, one could think that the composition of the ingestible composition is balanced in such a manner that the different ingredients interact in a metabolically optimised manner. However, the theory is not fully developed and should accordingly not be regarded as being binding to the invention.

The data obtained in this measurement are given in Table 2. The values for glucose and the ingestible composition are given in mmol/l. The measurements were performed using the procedures as described above.

| | | | 72 g food | 18 g food |
|---|---|---|---|---|
| Time | | | composition | composition |
| (min.) | 50 g glucoseⁱ | 22.9 g glucoseⁱⁱ | productⁱⁱⁱ | product^{iv} |
| 0 | 4.5 | 4.4 | 4.4 | 4.7 |
| 30 | 8.5 | 7.3 | 5.5 | 5.6 |
| 60 | 6.9 | 5.3 | 4.1 | 4.6 |
| 90 | 4.4 | 4.2 | 4.2 | 4.6 |
| 120 | 3.6 | 3.8 | 4.6 | 4.7 |

PAUC (= Positive Area Under Curve)

| | | | |
|---|---|---|---|
| 19.08 | 11.22 | 3.18 | 2.17 |

Glycemic Load vs. 50 g glucose

| | | |
|---|---|---|
| 100 | 16.7 | 11.3 |

Glycemic Load vs. 22.9 g glucose

| | | |
|---|---|---|
| 100 | 28.3 | 19.3 |

ⁱA test made on 50 g glucose
ⁱⁱA test made on 22.9 g glucose
ⁱⁱⁱA test made on 72 g food composition product
^{iv}A test made on 18 g food composition product

Various studies have since been performed where individuals with metabolic syndrome and its variants were placed on regimens with the above described ingestible composition. The studies showed an effective treatment, or at least amelioration, or metabolic syndrome in a statistically significant number of individuals. Each study involved packaging the ingestible composition into dry “packs” each containing about 18 grams of a ingestible composition containing the following ingredients: pea protein, whole egg powder, whey powder, dried apples, rose hip powder, egg albumin powder, ground sugar beet powder, milk protein, fructose, ascorbic acid.

Study Number 1

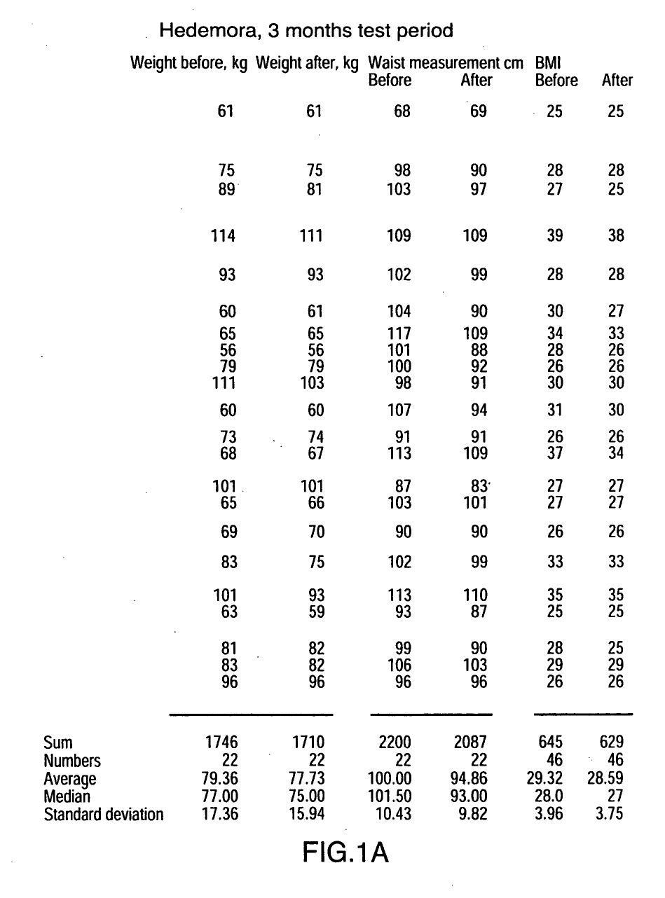

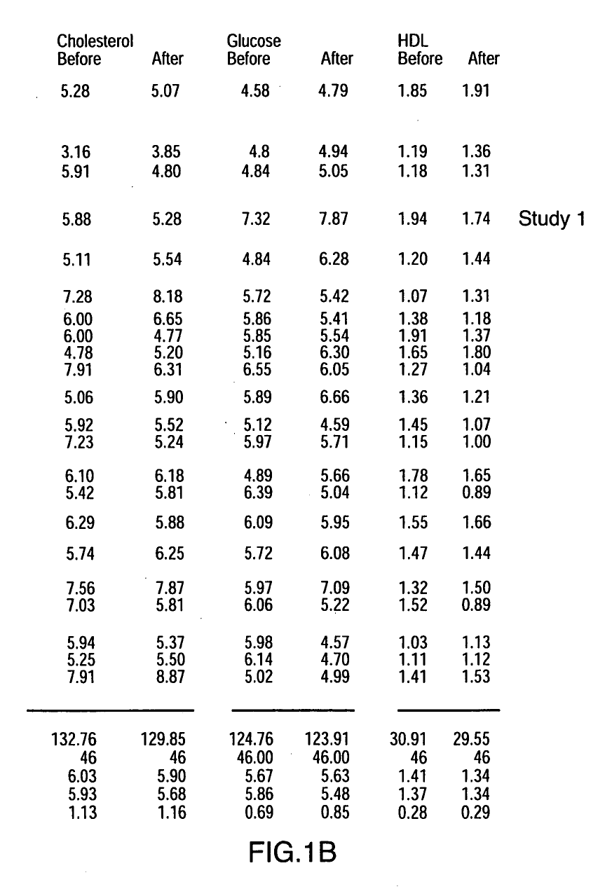

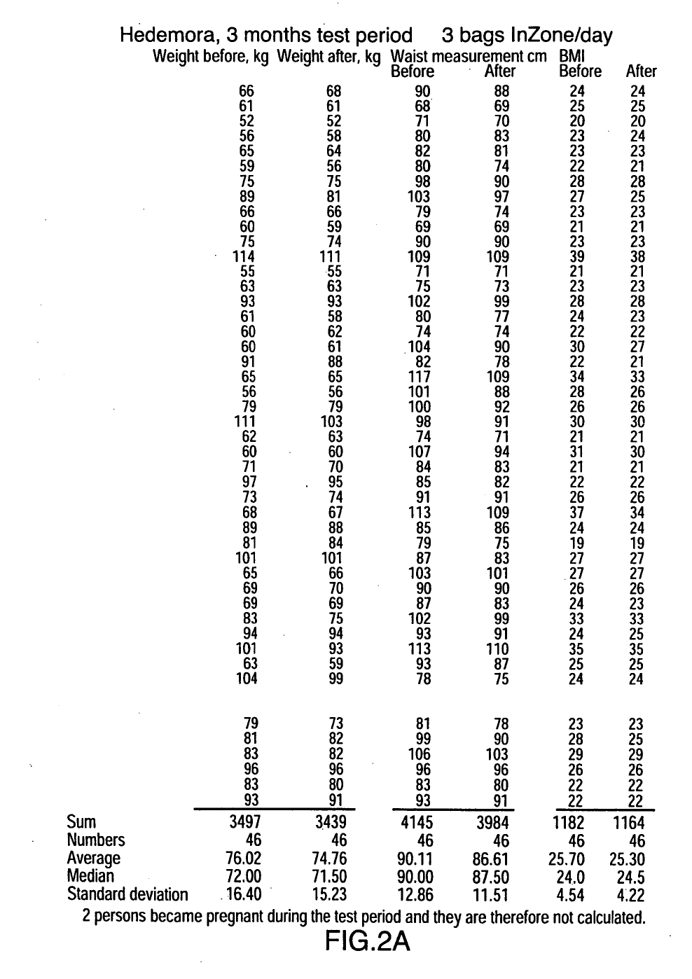

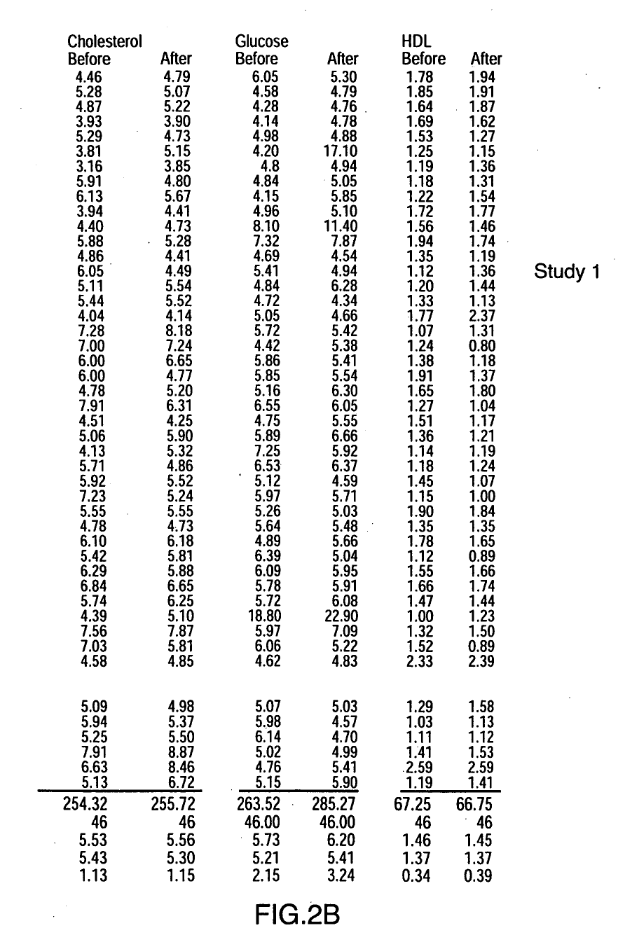

Study Number 1 was based on a test group of 46 persons. Each of these were asked to eat 2-3 packs of the ingestible composition per day. No other diet instructions were given. The study was run for three months and included running over the Christmas holidays. FIGS. 1A, 1B, 2A and 2B show a tabulation of the results of Study Number 1. FIGS. 1A and 1B are parts of the same table and are meant to be viewed together side by side; FIG. 1B being a continuation of the table of FIG. 1A. FIGS. 2A and 2B are parts of the same table and are meant to be viewed together side by side; FIG. 2B being a continuation of the table of FIG. 2A.

After three months the research persons had lost in mean 3.5 cm of their abdominal circumference measured at the umbilical level, and a mean weight of 1.26 kg of body weight. Twenty one of the persons had a body mass index (“BMI”) greater than about 25 (mean=29.3) when they were included in the study, and a mean umbilical circumference of 100.50. cm.

At the three month control, the BMI and umbilical circumference had diminished to X and Y respectively.

Study Number 2

Background

A second study was performed in a Swedish clinic specialized in treating the metabolic syndrome. The clinic has performed more than 85,000 tests on different patients during the years and has adopted a significant knowledge and expertise in treating this syndrome. Normal treatment is based on changing food habits. Within this study patients were asked to include the ingestible composition into the treatment program.

Study layout

Study Number 2 is based on the results from a test group of eight persons, all with the observed symptoms of metabolic syndrome. One of them was a diagnosed diabetic. The study lasted for fourteen days; the first week the patients were admitted to a clinic; the second week they were permitted to go home. Blood tests were taken daily throughout the first week. After the second week the patients were then tested one final time.

During the first week the following menu was served:

Breakfast: Yogurt, cereals and fresh berries, or oat porridge; a slice of bread including butter, ham cheese; one egg or herring; and vegetables.

Lunch 1—11:00: 2-3 packs of ingestible composition, two fruits and coffee or tea.

Lunch 2—15:00: 2-3 packs of ingestible composition, one fruit and coffee or tea.

Dinner: Meat or fish, vegetables, pasta or unpolished rice. Also included were fresh berries or fruits along with some whipped cream for dessert.

After three months the research persons had lost in mean 3.5 cm of their abdominal circumference measured at the umbilical level, and a mean weight of 1.26 kg of body weight. Twenty one of the persons had a body mass index (“BMI”) greater than about 25 (mean=29.3) when they were included in the study, and a mean umbilical circumference of 100.50. cm. During the second week of the study, patients were sent home and were asked to follow the protocol as per first week in the clinic.

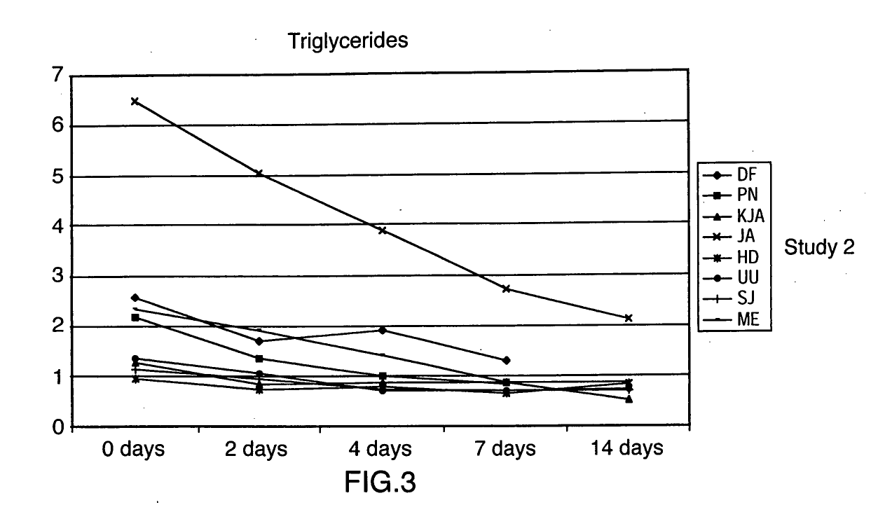

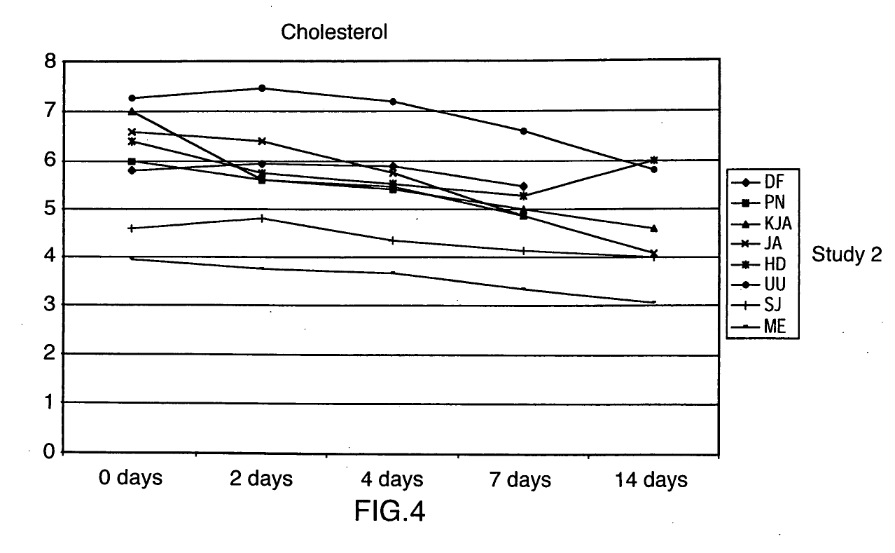

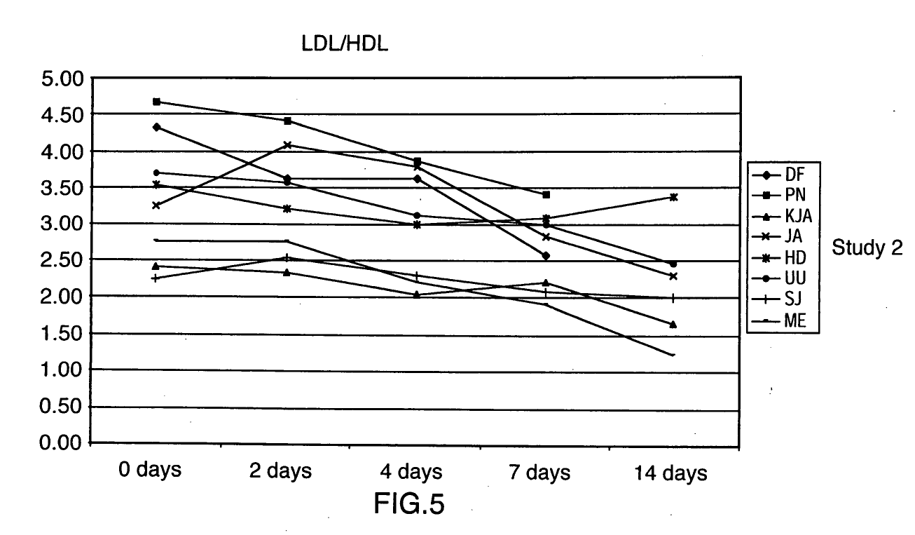

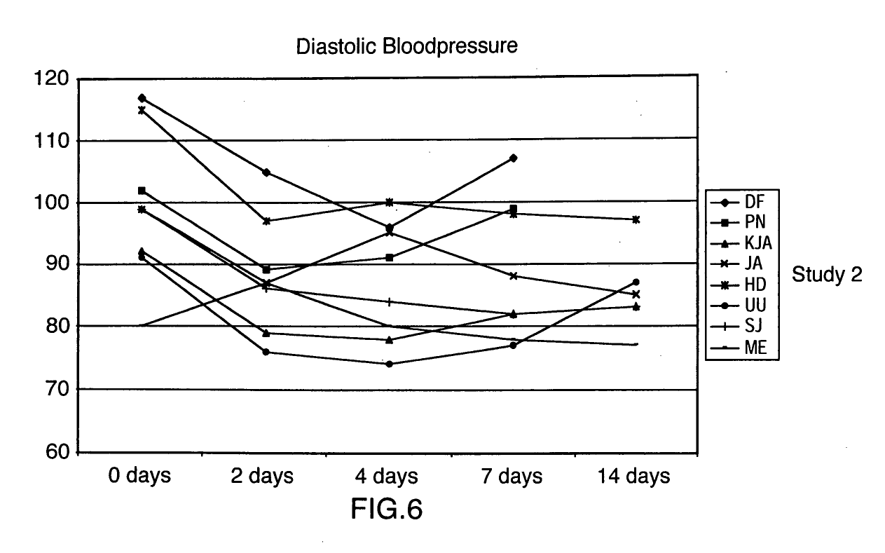

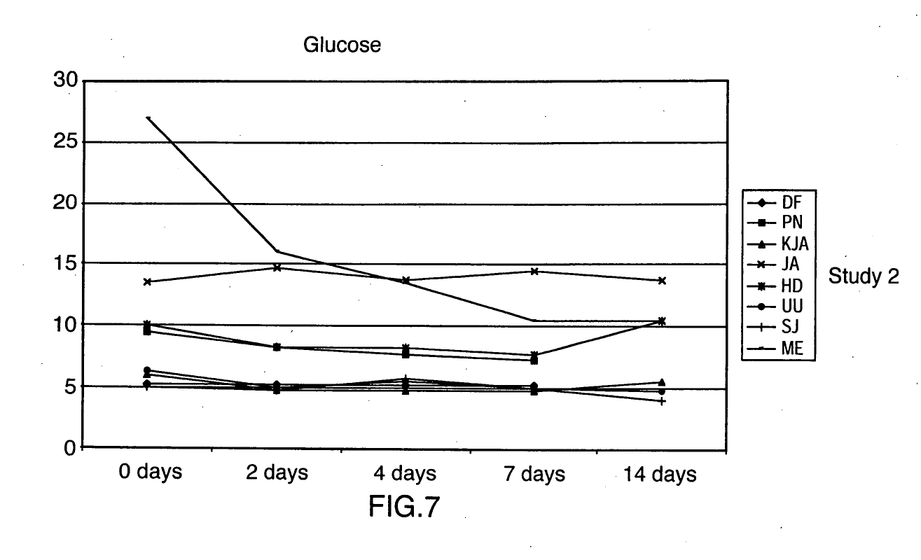

The study indicates that a low-glycemic loaded diet including a lunch meal divided into two ingestible composition servings can have a dramatic effect on the metabolic syndrome. The effects are also achievable while treating the patients with just ordinary food prescriptions, but the positive results reached including the ingestible composition in the treatment program are viewed by the inventors as being twice as effective—meaning the results being achieved under less than half of the normal time being necessary with just ordinary food prescriptions. Another positive proven effect with the ingestible composition treatment is that it is much more effective when the patients are being home in their normal environment and then just have to remember taking the adequate dosage of ingestible composition portions and can live their normal lives and eat more freely the standard meals, still maintaining the effects of a low GI-diet. FIGS. 3-7 show the results from the tests, wherein all of the biomarkers that can indicate metabolic syndrome and its variants show decreases over time when the treatment program is followed.

Study 3

In a third study, persons a little overweight were informed about the possibility of participating in a study with a new balanced meal supplement. If they were willing to test it a time was appointed in the morning when they come fasting. (Most persons have not very much time in the morning before they shall start to work, and therefore a simplified investigation was done. The body weight and height was measured and the body mass index calculated (body weight divided by height times height). The circumference of the abdomen at umbilicus level was measured as was the circumference around the hip and around the wrists and lean body mass calculated from a table. Then blood tests were taken for blood sugar, insulin, blood lipids, T3, T4, TSH, creatinine and liver enzymes as well as hemoglobin and serum iron. The persons were then served a breakfast after their wishes and instructions for how they can use the powder given. Half of the persons were randomized, to start with the ingestible composition immediately. After one month all returned to take -the same tests, and then the 50% non-randomized to ingestible composition are starting to take the ingestible composition and the ones being on powder stop to take it. After a further month final tests are taken on all persons. For certain patients with diabetes, obesity or other variant of metabolic syndrome, more time was taken for tests before they are included in the study. These tests include Åstrand test for measuring the oxygen uptake, electrocardiogram before and after the Åstrand test, a treadmill test and strength tests. All research persons are informed that it may take about two weeks before they fully accept the ingestible composition, and that they should patiently go on to take the ingestible composition at least two weeks before they give it up. So far no persons have given up taking the ingestible composition. Some persons complain about slight abdominal discomfort the first days on the ingestible composition, possibly due to the fiber content of the ingestible composition and that these persons have not been accustomed to eat food with fibre.

The first time the persons were involved in the study they only received instructions to take two packs each day. For patients with metabolic syndrome, or its variants such as diabetes or obesity, two packs at a time were recommended and at least two times a day. The time of the day during which the packs were taken was not specified. Some of the patients found it preferable to take two packs together with the breakfast, others to take two packs together with the lunch, or some take two packs at ten o'clock and two packs at two o'clock in the afternoon and eat all meals as usual, but eating less at each meal when taking the packs as mentioned. After participating in the program, the glycemic index for each of the patients was found to be very low. (i.e., Less than about 25 with an average of about 15)

Study 4

This study was set up using international standards how to measure the GI-value as set up Professor Jennie Brand-Miller, Sydney University Glycemic Index Research Service (SUGiRS) Australia. To determine a food's GI rating, measured portions of the food containing 10-50 grams of carbohydrate are fed to ten healthy people after an overnight fast. Finger-prick blood samples are taken at fifteen-thirty minute intervals over the next two hours. These blood samples are used to construct a blood sugar response curve for the two hour period. The area under the curve (AUC) is calculated to reflect the total rise in blood glucose levels after eating the test food. The GI rating (%) is calculated by dividing the AUC for the test food by the AUC for the reference food (same amount of glucose) and multiplying by one-hundred. The use of a standard food is for reducing the confounding influence of differences in the physical characteristics of the subjects. The average of the GI ratings from all ten subjects is published as the GI of that food.

The Glycemic Index (GI) of the Ingestible Composition

According to the international standards, the glycemic index on ten persons taking 158 g of the ingestible composition as one meal was measured. 158 g ingestible composition contains 50 g carbohydrates. (Approximately 8.77 packs, i.e., 158 g divided by 18 g.) The persons were asked to come fasting and take 158 g ingestible composition as their breakfast. The meal was fulfilled within fifteen minutes, and the ingestible composition was mixed with 800 ml of water. The blood sugar was measured every thirty minutes for two hours. The same persons took also 50 g of glucose on other days as breakfast. For each this was done three times on three different days both for the ingestible composition and glucose according to Brand-Miller's protocol. The area under curve for the blood sugar curve of 50 g glucose was defined as 100%. Ingestible composition had an extremely low glycemic index with a mean area under curve of 15.68%.

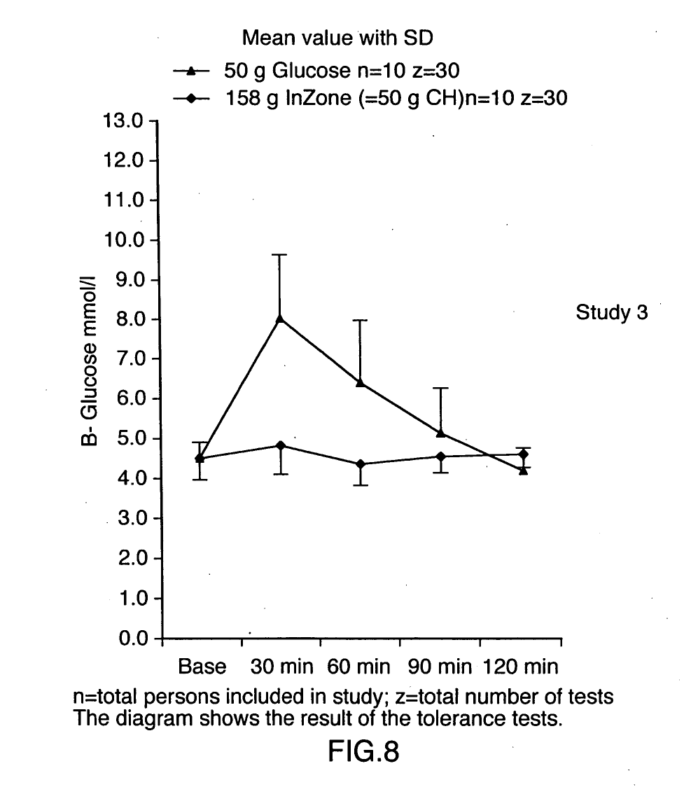

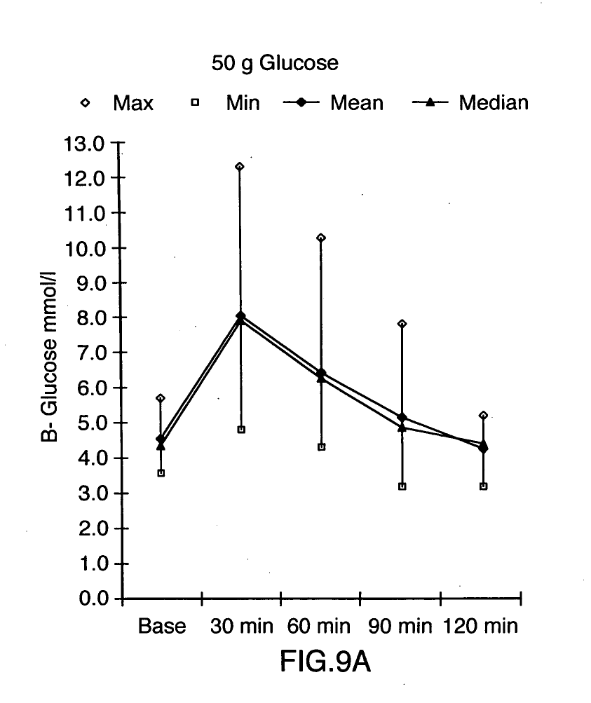

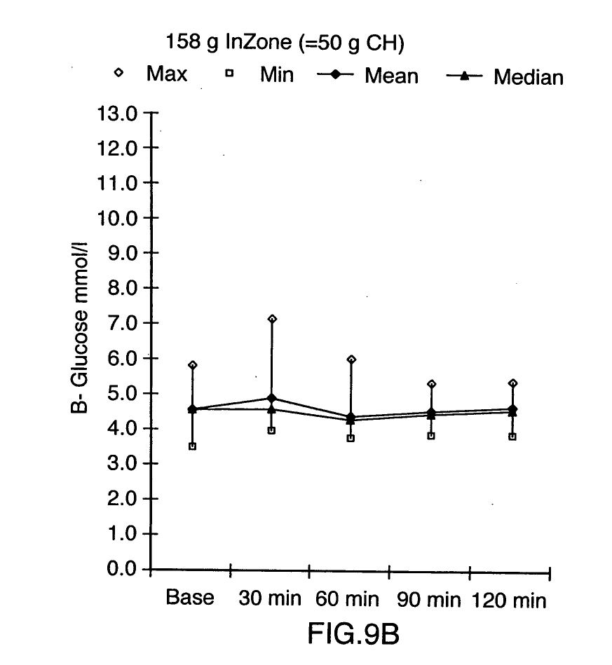

FIG. 8 shows the glycemic response to ingesting 50 gms of glucose while FIGS. 9A and 9B show the glycemic response to ingesting 158 gms of ingestible composition.

Study 5

One male patient complained of headaches, had a very high blood pressure, (220/120), was overweight, and a slightly pathological response on glucose test. The patient was instructed regarding his food habits and was taught to take two packs of the ingestible composition twice a day. He was also set on an angiotensine-2 blocker. The patient reported that the best time for him to take ingestible composition was at 10 AM and 2 PM. He kept all his other meals as usual. His craving for sugar disappeared and began to lose weight. After two months he hade lost twelve kg, bringing his weight to a normal range. His blood pressure fell to 115/75 and reported an excellent mood. His experience included being able to work efficiently after lunch the whole afternoon, without the need for a nap as had been required in the past. Sugar and sweet cravings were also reduced and overall food intake reduced. The patient did not exercise and did not change those habits during the study.

After three months he took 158 g ingestible composition (i.e., about 8.77 packages of 18 g each) in one meal and the blood sugar response was measured over the next two hours, his blood sugar increased with less than one mmol and he kept himself within a very narrow zone, in that the blood sugar remained substantially constant throughout the two-hour measurement period. This patient then stopped to take ingestible composition for one month. In that one-month period he regained three kg of weight and felt that his working power in the afternoons was reduced again. He then started with the powder again eating as he found best two packs at 10 AM and two packs at 2 PM and than he lost within the next month 3 kg and regained his normal weight and also his working power. The blood lipids of this patient dramatically changed to the better, he had high triglycerids and bad quote LDL/HDL (more than 6). Now he has normalized his triglycerids and has a quote LDL/HDL of 3.6.

While only specific combinations of the various features and components of the present invention have been discussed herein, it will be apparent to those of skill in the art that desired subsets of the disclosed features and components and/or alternative combinations of these features and components can be utilized, as desired. All third party sources cited herein are hereby incorporated by reference.

1. An ingestible composition comprising a plurality of components consisting of fat, carbohydrates, proteins, vitamins and minerals, wherein relative amounts of said components of said composition are chosen such that an intake of said product reduces the effects of metabolic syndrome.

2. The product according to claim 1, wherein the product weight ratio between carbohydrates, proteins and fat is of the magnitude of 0.8-2.0:1:0.1-0.4, respectively, wherein the carbohydrates are chosen from the group of foodstuffs having a low glycemic index.

3. The product according to claim 1, wherein the product weight ratio between carbohydrates, proteins and fat is of the magnitude of 0.8-2.0:1:0.1-0.3, respectively, wherein the carbohydrates are chosen from the group of foodstuffs having a low glycemic index.

4. The product according to claim 1 wherein the carbohydrates are derived from leguminous plants, preferably yellow peas, and rosaceous plants, preferably apples and rose hips.

5. The product according to claim 1 wherein at least 15% of the overall content is derived from yellow peas, at least 10% from apples, and at least 5% from rose hips.

6. The product according to claim 1 wherein the product is balanced so that the overall meal has GL-value below about 20,

7. The product according to claim 6 wherein said GL value is below about 10.

8. The product according to claim 1, wherein said carbohydrates are in the form of simple sugars from the group consisting of glucose, saccharose, fructose, maltose and lactose.

9. The product according to claim 8, wherein the sugars are present in a content of 2.1-2.5%, preferably about 2.3%, glucose, 1.8-2.2%, preferably about 2.0%, saccharose, 4.0-4.8%, preferably about 4.4% fructose, <0.03-0.05%, preferably about <0.04%, maltose and 12.9-15.7%, preferably about 14.3% lactose.

10. The product according to claim 1 wherein a ready-to-drink serving of said ingestible composition has either a low lactose content or alternatively is completely free from lactose.

11. The product according to claim 1 wherein it comprises 7-12% of dietary fibres.

12. The product according to claim 1 wherein the fat comprises the essential fatty acids with an omega-3 to omega-6 fatty acid ratio of 1:0.5 to 1:3.0, more preferably 1:0.8 to 1:2.0, and most preferably 1:1.0 to 1:1.3.

13. The product according to claim 1 wherein the fatty acids comprise 25-40%, saturated fatty acids, 30-45%, monounsaturated fatty acids, and 15-35%, di- and tri-unsaturated fatty acids.

14. The product according to claim 1 wherein the levels of docosahexaenoic acid (DHA) and eicosapentaenoic acid (EPA) are optimised and constitute between 1.9-2.3%, preferably about 2.1% of the total fat.

15. The product according to claim 1 wherein the whole egg content in the composition is supplied from especially fed chicken in order to give the whole egg product the preferred balance between saturated, monounsaturated and polyunsaturated fat, and the balance between omega-3 and omega-6 fatty acids in a rating of 1:0.7-1.5.

16. The product according to claim 1 wherein it comprises 20 amino acids including the 8 essential amino acids.

17. The product according to claim 1 wherein it comprises vitamins A, B, C, D, and E, and the essential minerals iron (Fe), zinc (Zn), calcium (Ca), phosphorus (P), magnesium (MG), copper (Cu), manganese (Mn), chromium (CR), selenium (Se), silica (Si), potassium (K), and sodium (Na), wherein potassium is present in high amounts, and sodium in small amounts, in physiological doses.

18. The product according to claim 1 having a Ph range of about 5.8 to about 6.2 and acts like a buffer.

19. The product according to claim 1 wherein it is gluten free.

20. The product according to claim 1 containing no artificial sweeteners, preservatives, flavorings, dyes or thickening agents.

21. The product according to claim 1 containing less than 75 kCal per portion serving, and correspondingly less than 425 kcal per 100 g in a powder mix dry form.

22. A method of treating metabolic syndrome comprising the step of consuming each day between about fifteen grams and about eighty grams of an ingestible composition comprising fat, carbohydrates, proteins, vitamins and minerals, wherein relative amounts of said components of said composition are chosen such that an intake of said product reduces the effects of metabolic syndrome.

23. An ingestible composition for use in treating metabolic syndrome comprising a plurality of components consisting of fat, carbohydrates, proteins, vitamins and minerals, wherein relative amounts of said components of said composition are chosen such that an intake of said product reduces the effects of metabolic syndrome.

24. An ingestible composition comprising syndrome comprising a plurality of components consisting of fat, carbohydrates, proteins, vitamins and minerals, wherein relative amounts of said components of said food composition product are chosen such that an intake of said product reduces the effects of metabolic syndrome, said composition used in the preparation of a medicament for the treatment of at least one of metabolic syndrome, diabetes, thrombosis, and obesity.

25. A method of treating at least one of metabolic syndrome, diabetes, and obesity comprising the step of consuming each day for at least a period of about two weeks between about fifteen grams and about eighty grams of an ingestible composition comprising: pea protein, whole egg powder, whey powder, dried apples, rose hip powder, egg albumin powder, ground sugar beet powder, milk protein, fructose, ascorbic acid.

26. A method of reducing thrombotic risk comprising the step of consuming each day for at least a period of about two weeks between about fifteen grams and about eighty grams of an ingestible composition comprising: pea protein, whole egg powder, whey powder, dried apples, rose hip powder, egg albumin powder, ground sugar beet powder, milk protein, fructose, ascorbic acid.
